(19) United States
(12) Patent Application Publication
(10) Pub. No.: US 2024/0417433 A1
(43) Pub. Date: Dec. 19, 2024

(54) Bispecific Binding Molecule

(71) Applicant: NANTONG YICHEN BIOPHARMA. CO. LTD., Nantong, Jiangsu (CN)

(72) Inventors: Feng WANG, Suzhou, Jiangsu (CN); Na CHEN, Suzhou, Jiangsu (CN)

(21) Appl. No.: 18/701,240

(22) PCT Filed: Oct. 15, 2022
(86) PCT No.: PCT/CN2022/125540

§ 371 Date: Apr. 14, 2024

(30) Foreign Application Priority Data
Oct. 15, 2021 (CN) 202111204907.6

(51) Int. Cl. C07K 14/435; A61K 39/00; A61P 1/00; C07K 16/28

(52) U.S. Cl. CPC C07K 14/43522; A61P 1/00; C07K 16/2839; A61K 2039/505; C07K 2317/31; C07K 2317/92; C07K 2319/30

(57) ABSTRACT

A bispecific binding molecule having high selectivity for Kv1.3 but different affinity for integrin β7, a pharmaceutical composition thereof, and a use thereof. The bispecific binding molecule is formed by the fusion of a toxin polypeptide that binds to a Kv1.3-type potassium ion channel and heavy chain or light chain variable region CDRs of an antibody that specifically binds to integrin β7. The obtained bispecific binding molecule has higher selectivity for Kv1.3^{high} β7^{high} T cells (relative to Kv1.3^{low} β7^{high} T cells), and can greatly improve the therapeutic efficacy, while preventing possible adverse reactions caused by other cells that positively express integrin β7. The described difference in binding activity toward two targets enables the bispecific binding molecule to have a binding preference for the two targets, thereby having certain advantages in the design of antibody drugs.

### CROSS-REFERENCES TO RELATED APPLICATION

The present application is a National Stage of International Patent Application No. PCT/CN2022/125540 filed on Oct. 15, 2022, which claims the benefit of the priority of the Chinese patent application with the application No. 202111204907.6, filed to the China National Intellectual Property Administration on Oct. 15, 2021, the entire content of which is incorporated in this application by reference.

### SEQUENCE LISTING

The instant application includes a Sequence Listing that has been submitted electronically in XML format and is hereby incorporated by reference in its entirety. The said XML file, named NTYC-0102-SEQ.xml, is 36,024 bytes in size. The sequence listing includes 26 sequences with SEQ ID NOs: 1 to 26, which are substantially identical in substance to the sequences disclosed in the PCT application. The sequence listing does not include any new matter.

### TECHNICAL FIELD

This application relates to a bispecific binding molecule, specifically a bispecific binding molecule with high selectivity for Kv1.3 and differing affinities for integrin β7.

### BACKGROUND

Over the past 20 years, with the development of genetic engineering technologies, over 50 different forms of bispecific binding molecules have been designed, such as IgG fusion, quadromas, diabodies, tandem single-chain variable fragments (ScFvs), DART, knobs-into-holes, DVD-Ig, etc. (Nat Rev Drug Discov 2019, 18 (8), 585-608; Drug Discov Today 2015, 20 (7), 838-847). Generally, bispecific binding molecules can be divided into two categories: IgG-based bispecific binding molecules, and non-IgG antibody types (Drug Discovery Today 2015, 20 (7), 838-847). Regardless of the type, It is necessary to link two antibody variable region fragments with binding and functional specificity by chemical or genetic engineering method to form a bispecific binding molecule in a framework. Currently, more than 30 bispecific binding molecules have entered clinical trials, but increasing evidence suggests that each framework form has its limitations (Drug Des Devel Ther 2018, 12, 195-208).

Inflammatory bowel disease (IBD) is a chronic inflammatory condition of the intestines associated with abnormal immune responses to intestinal bacteria. Alleviating intestinal tissue inflammation, restoring intestinal barrier function, and improving the balance of the gastrointestinal immune system are crucial for the cure of the disease (Inflamm Bowel Dis 2020, 26, 1131-1143). Although the pathogenesis of IBD is not fully understood, autoimmunity is believed to play a key role. Autoreactive memory T cells are considered a major cause of many autoimmune diseases, including multiple sclerosis (MS), type 1 diabetes (T1D), IBD, rheumatoid arthritis (RA), psoriasis, and systemic lupus erythematosus (SLE). Targeting autoreactive memory T cells has been considered a promising strategy for many immunomodulatory therapies for such diseases (Immunol Res 2013, 57, 12-22). The voltage-gated potassium channel Kv1.3 and the calcium-activated K+ channel KCa3.1 are essential for T cell membrane potential and calcium signaling and have been identified as potential therapeutic targets for immunosuppression. When Kv1.3 is activated, its expression is selectively upregulated on autoreactive memory T cells. Therefore, inhibiting Kv1.3 can selectively target pathogenic TEM cells in all T cell-mediated autoimmune diseases while avoiding immune responses associated with naive and central memory T (TCM) cells. Some animal toxins are natural inhibitors of the Kv1.3 channel with high selectivity and efficacy, showing potential to be developed into drugs to inhibit the activity of TEM cells. For example, ShK is a toxin derived from sea anemones that can specifically inhibit the Kv1.3 ion channel, and extensive research on ShK is currently ongoing (JBC 1998, 273, 32697-32707; Frontiers in neuroscience 2019, 13, 1393; PLOS ONE 2013, 8, e78712; Pharmacology 2009, 75, 762-773; PNAS 2013, 110, E2239). ShK-186, a synthetic analogue of ShK, has completed Phase I clinical trials for treatment of plaque psoriasis (PLOS ONE 2017, 12(7), e0180762). Another natural toxin, Vm24, extracted from Mexico scorpions, is also a highly selective and potent inhibitor of the Kv1.3 ion channel (Biochemistry 2012, 51, 4049-4061; Mol Pharmacol 2012, 82, 372-382). Experiments have shown that Vm24 can attenuate the response of CD4+ TEM cells to TCR stimulation (Cell Commun Signal: CCS 2018, 16, 45). However, these active peptides face many challenges when used directly as drugs, such as synthesis difficulties and short plasma half-lives (C. Beeton et al, PNAS 2006, 103, 17414; J Pharmacol Exp Ther 2012, 342, 642-653). Recently, the structure of the human Kv1.3 channel protein and its mutant H451N have been resolved, which will aid in the design and construction of Kv1.3 channel inhibitors (Cell Discov 2021, 7, 39).

Integrin β7 is an adhesion factor, and there is a ligand on the intestinal mucosa: mucosal address in cell adhesion molecule-1 (MAdCAM-1). The binding of β7 and MAdCAM-1 can mediate the migration and residence of lymphocytes to gut-associated lymphoid tissues. In the state of intestinal inflammation, the expression of MAdCAM-1 will increase, leading to more lymphocytes migrating to and residing in the inflamed tissues, exacerbating the inflammatory state of the lesion tissues and disease progression. Etrolizumab is a monoclonal antibody against β7, which is currently undergoing clinical trials as a therapeutic drug for inflammatory bowel disease (IBD). Adverse reactions reported from the phase II clinical trial of etrolizumab include rash, influenza-like illness, and joint pain (Brit J Dermatol 2021, 184 (2), 270-280). Phase III clinical results showed that the proportion of ulcerative colitis patients achieving remission with etrolizumab did not significantly differ from those achieving remission with placebos in two studies evaluating etrolizumab as induction therapy, while two studies evaluating etrolizumab as maintenance therapy failed to meet the primary endpoint (https://www.roche.com/media/releases/med-cor-2020-08-10.htm). These adverse reactions of anti-β7 antibody and its insufficient efficacy also herald the need to develop more selective drugs to effectively target TEM cells in IBD.

Therefore, there is a demand for bispecific binding molecules with stronger selectivity for Kv1.3 and tunable affinity for integrin β7, which can effectively reduce adverse reactions while ensuring efficacy.

### SUMMARY

Due to the significantly higher expression of integrin β7 on naïve T cells compared to that on TEM cells, antibodies specifically targeting integrin β7, such as etrolizumab, exhibit a more pronounced blocking effect on naïve T cells than on TEM cells. This may be the underlying reason for the limited efficacy of etrolizumab and its various adverse reactions. Our early experimental also showed that Kv1.3 inhibitors can effectively suppress the function of TEM cells and alleviate inflammatory response in the delayed-type hypersensitivity (DTH) model in rats. Therefore, it is extremely important to develop a bispecific binding molecule that targets integrin β7 and has higher selectivity for cells with high Kv1.3 expression to avoid or limit the side effects caused by other integrin β7-positive cells.

In the present application, by grafting Vm24, a Kv1.3-inhibiting toxin peptide, into the CDRs or FRs of the heavy chain and light chain variable regions of the integrin β7 antibody, we obtained bispecific binding molecules with higher affinity for Kv1.3 and differential affinity for integrin β7. By virtue of its high affinity and selectivity for Kv1.3, the bispecific binding molecules of the present application exhibits higher selectivity for Kv1.3 high β7 high T cells (relative to Kv1.3 low β7 high T cells), not only significantly enhancing therapeutic effect but also avoiding the possible adverse reactions caused by other integrin β7-positive cells. For this reason, the present application provides following technical solutions:

One aspect of the present application provides a bispecific binding molecule comprising: A) a toxin peptide that specifically binds to Kv1.3 type potassium ion channel; B) an antibody that specifically binds to integrin β7.

In some embodiments, the toxin peptide specifically binding to the Kv1.3 potassium channel is grafted into the CDRs (HCDR1, HCDR2, or HCDR3) of the heavy chain variable region of the integrin β7 antibody through a linker peptide. In one embodiment, the toxin peptide specifically binding to the Kv1.3 potassium channel is grafted into the CDRs (LCDR1, LCDR2, or LCDR3) of the light chain variable region of the integrin β7 antibody through a linker peptide.

In one embodiment, the toxin peptide specifically binding to the Kv1.3 potassium channel is grafted into the FRs (FR1H, FR2H, FR3H, or FR4H) of the heavy chain variable region of the integrin β7 antibody.

In one embodiment, the toxin peptide specifically binding to the Kv1.3 potassium channel is grafted into the FRs (FR1L, FR2L, FR3L, or FR4L) of the light chain variable region of the integrin β7 antibody.

In one embodiment, the toxin peptide specifically binding to the Kv1.3 potassium channel is Vm24. In one embodiment, the toxin peptide Vm24 has the amino acid sequence as shown in SEQ ID NO: 9. In one embodiment, the toxin peptide has an amino acid sequence with 95% homology to SEQ ID NO: 9.

In one embodiment, the antibody specifically binding to integrin β7 has LCDR1, LCDR2, LCDR3 sequences as shown in SEQ ID NO: 10, SEQ ID NO: 11, and SEQ ID NO: 12, respectively, and HCDR1, HCDR2, HCDR3 sequences as shown in SEQ ID NO: 13, SEQ ID NO: 14, and SEQ ID NO: 15, respectively.

In some embodiments, the bispecific binding molecule comprises: a) a toxin peptide specifically binds to the Kv1.3 potassium channel with the amino acid sequence as shown in SEQ ID NO: 9; b) an antibody specifically binding to integrin β7 with LCDR1, LCDR2, LCDR3, HCDR1, HCDR2, HCDR3 as shown in SEQ ID NO: 10, SEQ ID NO: 11, SEQ ID NO: 12, SEQ ID NO: 13, SEQ ID NO: 14, and SEQ ID NO: 15, respectively.

In some embodiments, the toxin peptide as shown in SEQ ID NO: 9 is grafted into the LCDR1, LCDR2, LCDR3, HCDR1, HCDR2, HCDR3, FR1L, FR2L, FR3L, FR4L, FR1H, FR2H, FR3H, or FR4H of the integrin β7 antibody.

In some embodiments, the linker peptide has the amino acid sequences as shown in

(SEQ ID NO: 16)
GGSGAKLAALKAKLAALKGGGGS
and/or
(SEQ ID NO: 17)
GGGGSELAALEAELAALEAGGSG.

In one embodiment, the toxin peptide as shown in SEQ ID NO: 9 is grafted into the LCDR1 of the integrin β7 antibody. In a specific embodiment, the toxin peptide as shown in SEQ ID NO: 9 is grafted between S28 and L32 of the LCDR1 of the integrin β7 antibody. In a specific embodiment, the toxin peptide as shown in SEQ ID NO: 9 is grafted between S28 and L32 of the LCDR1 of the integrin β7 antibody using the linker peptides as shown in SEQ ID NO: 16 and SEQ ID NO: 17. In one embodiment, the bispecific binding molecule has the HC as shown in SEQ ID NO: 1 and the LC as shown in SEQ ID NO: 3. In such embodiment, the bispecific binding molecule ATF-L1 has the following characteristics: its affinity for Kv1.3 is substantially similar to that of Syn-Vm24-LCDR3; its affinity for integrin β7 is 167.1 pM, approximately 10 times weaker than that of the integrin β7 antibody (17.57 pM); its blocking effect on interaction between β7high T cells and MAdCAM-1 (IC50=1.09 nM) is about 3 times weaker than that of the integrin β7 antibody (0.42 nM); its inhibitory effect on Kv1.3 current is similar as that of the integrin β7 antibody. Furthermore, in binding selectivity experiments, the percentage of antibody bound to Kv1.3highβ7high T cells is 2.4±0.1, 1.7±0.2, 1.3±0.1, and 1.2±0.1 times of antibody bound to Kv1.3lowβ7high T cells when the bispecific binding molecule is at the concentrations of 50 pM, 100 pM, 200 pM, or 400 pM, respectively; In competition experiments, Kv1.3lowβ7high T cells-bound ATF-L1-PE is more easily competed by etrolizumab (compared to Kv1.3highβ7high T cells), indicating that the bispecific binding molecule ATF-L1 has a higher selectivity for Kv1.3 than for β7, making it more likely to bind to T cells with high Kv1.3 expression; In the DSS mouse model, the bispecific binding molecule ATF-L1 can specifically inhibit the migration of human T cells with high Kv1.3 expression to inflamed intestinal tissues.

In one embodiment, the toxin peptide as shown in SEQ ID NO: 9 is grafted into the LCDR3 of the integrin β7 antibody. In a specific embodiment, the toxin peptide as shown in SEQ ID NO: 9 is grafted between 91G and 96N of the LCDR3 of the integrin β7 antibody. In a specific embodiment, the toxin peptide as shown in SEQ ID NO: 9 is grafted between 91G and 96N of the LCDR3 of the integrin β7 antibody using the linker peptides as shown in SEQ ID NO: 16 and SEQ ID NO: 17. In one embodiment, the bispecific binding molecule has the HC as shown in SEQ ID NO: 1 and the LC as shown in SEQ ID NO: 5. In such embodiment, the affinity of the bispecific binding molecule ATF-L3 to Kv1.3 is substantially similar to that of Syn-Vm24-LCDR3, and its affinity to integrin β7 (40.82 pM) is 2.3 times weaker than that of the integrin β7 antibody (17.57 pM).

In one embodiment, the toxin peptide as shown in SEQ ID NO: 9 is grafted into the HCDR1 of the integrin β7 antibody. In a specific embodiment, the toxin peptide as shown in SEQ ID NO: 9 is grafted between 26F and 32N of the HCDR1 of the integrin β7 antibody. In a specific embodiment, the toxin peptide as shown in SEQ ID NO: 9 is grafted between 26F and 32N of the HCDR1 of the integrin β7 antibody using the linker peptides as shown in SEQ ID NO: 16 and SEQ ID NO: 17. In one embodiment, the bispecific binding molecule has the HC as shown in SEQ ID NO: 6 and the LC as shown in SEQ ID NO: 2. In such embodiment, the affinity of the bispecific binding molecule ATF-H1 for Kv1.3 is substantially similar to that of Syn-Vm24-LCDR3, and its inhibitory effect on Kv1.3 potassium current (0.72 nM) is also similar to that of Syn-Vm24-LCDR3 (0.49 nM).

In one embodiment, the toxin peptide as shown in SEQ ID NO: 9 is grafted into the HCDR3 of the integrin β7 antibody. In a specific embodiment, the toxin peptide as shown in SEQ ID NO: 9 is grafted between 97R and 102G of the HCDR3 of the integrin β7 antibody. In a specific embodiment, the toxin peptide as shown in SEQ ID NO: 9 is grafted between 97R and 102G of the HCDR3 of the integrin β7 antibody using the linker peptides as shown in SEQ ID NO: 16 and SEQ ID NO: 17. In one embodiment, the bispecific binding molecule has the HC as shown in SEQ ID NO: 8 and the LC as shown in SEQ ID NO: 2. In such embodiment, the affinity of the bispecific binding molecule ATF-H3 for Kv1.3 is substantially similar to that of Syn-Vm24-LCDR3, and its affinity(13.88 pM) for integrin β7 is also similar to that of the integrin β7 antibody (17.57 pM).

In one embodiment, the toxin peptide as shown in SEQ ID NO: 9 is grafted into the FR3L of the integrin β7 antibody. In a specific embodiment, the toxin peptide as shown in SEQ ID NO: 9 is grafted between 56S and 60S of the FR3L of the integrin β7 antibody. In a specific embodiment, the toxin peptide as shown in SEQ ID NO: 9 is grafted between 56S and 60S of the FR3L of the integrin β7 antibody using the linker peptides as shown in SEQ ID NO: 16 and SEQ ID NO: 17. In one embodiment, the bispecific binding molecule has the HC as shown in SEQ ID NO: 1 and the LC as shown in SEQ ID NO: 4. In such embodiment, the affinity of bispecific binding molecule ATF-L2 for Kv1.3 is similar to that of Syn-Vm24-LCDR3, and its affinity (35.9 pM) for integrin β7 is approximately 2 times weaker than that of the integrin β7 antibody (17.57 pM).

In one embodiment, the toxin peptide as shown in SEQ ID NO: 9 is grafted into the FR3H of the integrin β7 antibody. In a specific embodiment, the toxin peptide as shown in SEQ ID NO: 9 is grafted between 60N and 65S of the FR3H of the integrin β7 antibody. In a specific embodiment, the toxin peptide as shown in SEQ ID NO: 9 is grafted between 60N and 65S of the FR3H of the integrin β7 antibody using the linker peptides as shown in SEQ ID NO: 16 and SEQ ID NO: 17. In one embodiment, the bispecific binding molecule has the HC as shown in SEQ ID NO: 7 and the LC as shown in SEQ ID NO: 2. In such embodiments, the affinity of bispecific binding molecule ATF-H2 for Kv1.3 is similar to that of Syn-Vm24-LCDR3, and its affinity(19.25 pM) for integrin β7 is also similar to that of the integrin β7 antibody (17.57 pM); furthermore, its inhibitory effect on Kv1.3 current (0.53 pM) is similar to that of Syn-Vm24-LCDR3 (0.49 pM), and its inhibitory effect on the adhesion of β7high T cells to MAdCAM-1 (0.45 pM) is also similar to that of the integrin β7 antibody (0.42 pM).

Another aspect of the present application provides a polynucleotide encoding the aforementioned bispecific binding molecules.

Another aspect of the present application provides a pharmaceutical composition comprising the aforementioned bispecific binding molecules and a pharmaceutically acceptable carrier.

Another aspect of the present application provides the use of the aforementioned bispecific binding molecule in the preparation of drugs for the treatment or prevention of diseases related to the Kv1.3 potassium channel and/or integrin β7. In some embodiments, diseases related to the Kv1.3 potassium channel and/or integrin β7 include: inflammatory diseases, immune and proliferative diseases, rheumatoid arthritis (RA), ankylosing spondylitis, psoriatic arthritis, osteoarthritis, osteoporosis, uveitis, inflammatory fibrosis, scleroderma, pulmonary fibrosis, cirrhosis, inflammatory bowel disease, Crohn's disease, ulcerative colitis, asthma, allergic asthma, allergies, chronic obstructive pulmonary disease (COPD), multiple sclerosis, psoriasis, contact dermatitis, systemic lupus erythematosus (SLE) and other forms of lupus, diabetes, type I diabetes, obesity, cancer, lupus, restenosis, systemic sclerosis, scleroderma, glomerulonephritis, Sjögren's syndrome, inflammatory bone resorption, transplant rejection or graft-versus-host disease, etc.

### BRIEF DESCRIPTION OF THE DRAWINGS

FIG. 1 shows the SDS-PAGE of the antibody fusion protein of Vm24 and etrolizumab.

FIG. 2A presents the ELISA results of different bispecific binding molecules binding to integrin β7.

FIG. 3 displays the flow cytometry results of high levels of β7 on human T cells induced by PMA and ionomycin (IM).

FIG. 4 illustrates the inhibitory effect of bispecific binding molecules on the adhesion between integrin β7 T cells and MAdCAM-1 detected by cell adhesion assay.

FIG. 5 demonstrates the inhibitory effect of bispecific binding molecules on Kv1.3 potassium currents by the Flipr real-time fluorescence detection method.

FIG. 6 shows the flow cytometry results of Jurkat cells infected by lentivirus with high expression of Kv1.3 and EGFP genes, wherein A) depicts a schematic diagram of the lentiviral plasmid construction; B) shows the negative control group (the rate of Kv1.3-positive cells is close to 0) without anti-Kv1.3 antibody; C) indicates the rate of Kv1.3-positive cell when the EGFP-positivity rate is 96.74%; D) shows the rate of Kv1.3-positive cell when the EGFP positivity rate is 84.57%; E) displays the rate of Kv1.3-positive cell when the EGFP positivity rate is 20.52%.

FIG. 7 illustrates the binding selectivity of bispecific binding molecules on different subsets of human T cells. A presents the flow cytometry results where the left two columns show the migration rates (PE fluorescence positivity rate) of ATF-L1-PE binding to Kv1.3highβ7high T cells (EGFP-positive cells) or Kv1.3lowβ7high T cells (EGFP-negative cells) at different concentrations, and the right two columns show the migration rates (PE fluorescence positivity rate) of ATF-L1-PE binding to Kv1.3highβ7high T cells or Kv1.3lowβ7high T cells when monoclonal antibody etrolizumab with concentration 10 times that of ATF-L1-PE is added; B quantitatively analyzes the ratio of the migration rates of ATF-L1 binding to Kv1.3highβ7high T cells or Kv1.3lowβ7high T cells in binding experiments and competitive binding experiments.

FIG. 8 presents in vivo activity detection of bispecific binding molecules; A depicts the proportion of adoptively transferred human T cells in the blood of DSS model animals and normal control animals detected by flow cytometry; B displays the quantitative statistics and analysis of human T cells in the blood of each group detected by flow cytometry; C provides statistical analysis of the in vivo two-photon imaging results of colon tissue.

FIG. 9 displays the imaging of mouse colon tissue slices, where A shows HE staining of colon tissue from the water-drinking normal control group, demonstrating intact villous structures, goblet cells (*), and crypt structures (#); B displays HE staining of the DSS group showing disrupted villous tissue structure, significant loss of goblet cells, and crypt structure loss; C presents statistical analysis results of cells expressing high levels of Kv1.3.

FIG. 10 shows the body weight changes of mice in each group during DSS modeling. The two doses of ATF-L1 (low-dose group and high-dose group) and the high-dose group of Etrolizumab significantly improved the weight loss induced by DSS in mice. Significant analysis of body weight between groups on the 9th day: ATF-L1 low-dose group Vs DSS group (P<0.01, **); ATF-L1 high-dose group Vs DSS group (P<0.001, ***); Etro high-dose group Vs DSS group (P<0.01, **); DPBS blank control group VS DSS group (P<0.001, ***), where “etro” refers to etrolizumab.

FIG. 11 presents the Disease Activity Index (DAI) scores of mice in each group during DSS.

FIG. 12 is the comparison of colon tissue lengths of mice in each group during DSS.

### DETAILED DESCRIPTION OF THE EMBODIMENTS

This application is herein described in detail through reference to the following definitions and embodiments. Contents of patents and publications mentioned herein, including all sequences disclosed in such patents and publications, are explicitly incorporated herein by reference.

The term “polypeptide” refers to a molecule comprising at least two amino acid residues connected by peptide bonds to form a peptide. Polypeptides can also be referred to as “proteins”.

As used herein, the term “inhibitory toxin polypeptide targeting Kv1.3 potassium ion channel” refers to a polypeptide that can specifically bind to the Kv1.3 potassium ion channel and inhibit its activity. In the present application, the “inhibitory polypeptide targeting Kv1.3 potassium ion channel” preferably refers to a novel polypeptide Vm24 isolated from the venom of the Mexican scorpion V. mexicanus. In a preferred embodiment, Vm24 has the amino acid sequence shown in SEQ ID NO: 9. Amino acid sequences having at least 90%, 95%, 96%, 97%, 98%, and 99% identity with SEQ ID NO: 9, and being capable of inhibiting Kv1.3 type potassium ion channels are also included in the present application.

The “bispecific binding molecule” of the present application can specifically bind to both the Kv1.3 potassium ion channel and integrin β7.

Grafting exogenous peptides into the CDRs region of an antibody often disrupts the binding of the antibody to its antigen, leading to a significant decrease or even loss of affinity of the antibody. Typically, not all of the six CDRs of antibodies directly interact with antigens. We reason that if Vm24 is grafted into the CDRs that do not interact with the antigen, it is possible to retain the binding of the antibody to its cognate antigen while gain the new functionality of Vm24, which affords a new bispecific antibody by design. To this end, Vm24 was grafted into heavy chain variable region or light chain CDRs of the integrin β7 antibody, or their vicinity, through a coiled-coil-GS linker peptide.

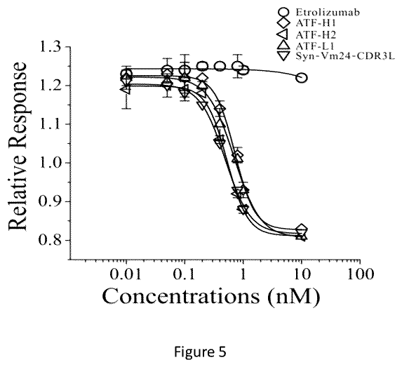

In a preferred embodiment, Vm24 is grafted between 28S and 32S of the light chain variable region CDR1 of the integrin β7 antibody through the coiled-coil-GS linker peptide. The resulting bispecific molecule ATF-L1 has a weaker affinity (167.10 pM) for integrin β7 than that of the integrin β7 antibody (17.57 pM), and similar affinity for Kv1.3 as that of Syn-Vm24-LCDR3. In a further embodiment, the inhibitory effect of ATF-H1 (IC50=1.09 nM) on the adhesion of β7-high T cells to MAdCAM-1 is similar to that of the integrin β7 antibody (IC50=0.42 nM) (FIG. 4), while the inhibitory effect on Kv1.3 current is similar to that of Syn-vm24-LCDR3(FIG. 5).

In a preferred embodiment, Vm24 is grafted between the last 26S and 32N of the heavy chain variable region CDR1 of the integrin β7 antibody through a coiled-coil-GS linker peptide. The affinity of the resulting bispecific molecule ATF-H1 is weaker than that of the integrin β7 antibody, and its affinity (IC50=0.72 nM) for Kv1.3 is equivalent to that of Syn-Vm24-LCDR3 (IC50=0.49 nM) (FIG. 4) reported in literature (PNAS 2016,113(41):11501-11506). In further embodiments, ATF-H1 shows minimal inhibitory effect on the adhesion of β7-high T cells to MAdCAM-1 while displaying similar inhibitory effect on Kv1.3 current as that of Syn-Vm24-LCDR3.

In a preferred embodiment, Vm24 is grafted between 60N and 65R of the heavy chain variable region FR3 of the integrin β7 antibody through a coiled-coil-GS linker peptide. The resulting bispecific molecule ATF-H2(19.25 pM) has similar affinity for integrin β7 to that of the integrin β7 antibody (17.57 pM), and also has similar affinity for Kv1.3 as that of Syn-Vm24-LCDR3 reported in literature (PNAS 2016,113(41):11501-11506). In further embodiments, ATF-H1 (IC50=1.09 nM) shows similar inhibitory effect on the adhesion of β7-high T cells to MAdCAM-1 as that of the integrin β7 antibody (IC50=0.42 nM) and a similar inhibitory effect on Kv1.3 current.

In a preferred embodiment, Vm24 is grafted between 56S and 60S of the light chain variable region FR3 of the integrin β7 antibody through a coiled-coil-GS linker peptide. The resulting bispecific molecule ATF-L2 has a slightly weaker affinity (35.9 pM) for integrin β7 than that of the integrin β7 antibody (17.57 pM) and a similar affinity for Kv1.3 as that of Syn-Vm24-LCDR3 reported in literature (PNAS 2016, 113(41):11501-11506).

In a preferred embodiment, Vm24 is grafted between 91G and 96N of the light chain variable region CDR3 of the integrin β7 antibody through a coiled-coil-GS linker peptide. The resulting bispecific molecule ATF-L3 has a weaker affinity (40.82 pM) for integrin β7 than that of the integrin β7 antibody (17.57 pM), and a similar affinity for Kv1.3 as that of Syn-Vm24-LCDR3 reported in literature (PNAS 2016, 113(41):11501-11506).

### EXAMPLES

### 1. Design and Construction of Bispecific Binding Molecules

Vm24 is a peptide composed of 36 amino acid residues, with a molecular weight of 3864 Da. The amino acid sequence is: AAAISCVGSPECPPKCRAQGCKNGKCMNRKCKCYYC (SEQ ID NO: 9), and the DNA sequence is:

(SEQ ID NO: 26)
gccgctgcaatctcctgcgtcggcagccccgaatgtcctcccaagtgccg
ggctcagggatgcaagaacggcaagtgtatgaaccggaagtgcaagtgct
actattgc.

The DNA sequence of complementarity-determining region 1 of Etrolizumab light chain (LCDR1) is gag age gtg gac gac ctg, corresponding to the amino acid sequence 27ESVDDL32. The LCDR2 DNA sequence is aag tac gcc age cag, with corresponding amino acid sequence 49KYASQ53. The LCDR3 DNA sequence is cag cag ggc aac age ctg ccc aac acc, with corresponding amino acid sequence 89QQGNSLPNT97. The DNA sequence of complementarity-determining region 1 of Etrolizumab heavy chain (HCDR1) is TTC TTC ATC ACC AAC AAC, with corresponding amino acid sequence 27FFITNN32. The HCDR2 DNA sequence is ATC AGC TAC AGC GGC AGC ACC, with corresponding amino acid sequence 51ISYSGST57. The HCDR3 DNA sequence is GCC CGC ACC GGC AGC AGC GGC TAC TTC GAC, with corresponding amino acid sequence 96ARTGSSGYFD105.

Vm24 is grafted into CDRs of both the light and heavy chains of etrolizumab, with coiled-coil-GS linkers at both ends (upstream amino acid sequence: GGSGAKLAALKAKLAALKGGGGS; downstream amino acid sequence: GGGGSELAALEAELAALEAGGSG) connecting to the antibody amino acid sequences. The resulting sequences of the bispecific binding molecules are as follows:

Etrolizumab HC (SEQ ID NO: 1):
EVQLVESGGGLVQPGGSLRLSCAASGFFITNNYWGWVRQAPGKGLEWVGYISYSGSTSYNPSLKSRFT
ISRDTSKNTFYLQMNSLRAEDTAVYYCARTGSSGYFDFWGQGTLVTVSSASTKGPSVFPLAPSSKSTSG
GTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKF
NWYVDGVEVHNAKTKPREEQYASTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKL
TVDKSRWQQGNVFSCSVMHEALHNHYTQKSLSLSPGK
Etrolizumab LC (SEQ ID NO: 2):
DIQMTQSPSSLSASVGDRVTITCRASESVDDLLHWYQQKPGKAPKLLIKYASQSISGVPSRFSGSGSGT
DFTLTISSLQPEDFATYYCQQGNSLPNTFGQGTKVEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTK
SFNRGEC
ATF-L1 LC (SEQ ID NO: 3):
DIQMTQSPSSLSASVGDRVTITCRASESGGSGAKLAALKAKLAALKGGGGSAAAISCVGSPECPPKCR
AQGCKNGKCMNRKCKCYYCGGGGSELAALEAELAALEAGGSGLLHWYQQKPGKAPKLLIKYASQSI
SGVPSRFSGSGSGTDFTLTISSLQPEDFATYYCQQGNSLPNTFGQGTKVEIKRTVAAPSVFIFPPSDEQLK
SGTASVVCLLNNFYPREAKVQWKVDNALQSGNSQESVTEQDSKDSTYSLSSTLTLSKADYEKHKVYA
CEVTHQGLSSPVTKSFNRGEC
ATF-L2 LC (SEQ ID NO: 4):
DIQMTQSPSSLSASVGDRVTITCRASESVDDLLHWYQQKPGKAPKLLIKYASQSISGGSGAKLAALKA
KLAALKGGGGSAAAISCVGSPECPPKCRAQGCKNGKCMNRKCKCYYCGGGGSELAALEAELAALEA
GGSGSRFSGSGSGTDFTLTISSLQPEDFATYYCQQGNSLPNTFGQGTKVEIKRTVAAPSVFIFPPSDEQLK
SGTASVVCLLNNFYPREAKVQWKVDNALQSGNSQESVTEQDSKDSTYSLSSTLTLSKADYEKHKVYA
CEVTHQGLSSPVTKSFNRGEC
ATF-L3 LC (SEQ ID NO: 5):
DIQMTQSPSSLSASVGDRVTITCRASESVDDLLHWYQQKPGKAPKLLIKYASQSISGVPSRFSGSGSGT
DFTLTISSLQPEDFATYYCQQGGGSGAKLAALKAKLAALKGGGGSAAAISCVGSPECPPKCRAQGCKN
GKCMNRKCKCYYCGGGGSELAALEAELAALEAGGSGNTFGQGTKVEIKRTVAAPSVFIFPPSDEQLKS
GTASVVCLLNNFYPREAKVQWKVDNALQSGNSQESVTEQDSKDSTYSLSSTLTLSKADYEKHKVYAC
EVTHQGLSSPVTKSFNRGEC
ATF-H1 HC (SEQ ID NO: 6):
EVQLVESGGGLVQPGGSLRLSCAASGGGSGAKLAALKAKLAALKGGGGSAAAISCVGSPECPPKCRA
QGCKNGKCMNRKCKCYYCGGGGSELAALEAELAALEAGGSGYWGWVRQAPGKGLEWVGYISYSG
STSYNPSLKSRFTISRDTSKNTFYLQMNSLRAEDTAVYYCARTGSSGYFDFWGQGTLVTVSSASTKGPS
VFPLAPSSKSTSGGTAALGCLVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFPPKPKDTLMISRTPEVTCVV
VDVSHEDPEVKFNWYVDGVEVHNAKTKPREEQYASTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
ATF-H2 HC (SEQ ID NO: 7):
EVQLVESGGGLVQPGGSLRLSCAASGFFITNNYWGWVRQAPGKGLEWVGYISYSGSTSYNGGSGAKL
AALKAKLAALKGGGGSAAAISCVGSPECPPKCRAQGCKNGKCMNRKCKCYYCGGGGSELAALEAEL
AALEAGGSGSRFTISRDTSKNTFYLQMNSLRAEDTAVYYCARTGSSGYFDFWGQGTLVTVSSASTKGP
SVFPLAPSSKSTSGGTAALGCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFPPKPKDTLMISRTPEVTCV
VVDVSHEDPEVKFNWYVDGVEVHNAKTKPREEQYASTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
ATF-H3 HC (SEQ ID NO: 8):
EVQLVESGGGLVQPGGSLRLSCAASGFFITNNYWGWVRQAPGKGLEWVGYISYSGSTSYNPSLKSRFT
ISRDTSKNTFYLQMNSLRAEDTAVYYCARGGSGAKLAALKAKLAALKGGGGSAAAISCVGSPECPPK
CRAQGCKNGKCMNRKCKCYYCGGGGSELAALEAELAALEAGGSGGYFDFWGQGTLVTVSSASTKG
PSVFPLAPSSKSTSGGTAALGCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYASTYRVVSVLTVLHQDWLNGKEYKCKVSNK
ALPAPIEKTISKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
Vm24 (SEQ ID NO: 9): AAAISCVGSPECPPKCRAQGCKNGKCMNRKCKCYYC
LCDR1 (SEQ ID NO: 10): ²⁷ESVDDL³²
LCDR2 (SEQ ID NO: 11): ⁴⁹KYASQ⁵³
LCDR3 (SEQ ID NO: 12): ⁸⁹QQGNSLPNT⁹⁷
HCDR1 (SEQ ID NO: 13): ²⁷FFITNN³²
HCDR2 (SEQ ID NO: 14): ⁵¹ISYSGST⁵⁷
HCDR3(SEQ ID NO: 15): ⁹⁶ARTGSSGYFD¹⁰⁵
Linker 1: GGSGAKLAALKAKLAALKGGGGS (SEQ ID NO: 16)
Linker2: GGGGSELAALEAELAALEAGGSG (SEQ ID NO: 17)
Etrolizumab HC (SEQ ID NO: 18) Nucleotide sequence:
GAGGTGCAGCTGGTGGAGAGCGGCGGCGGCCTGGTGCAGCCCGGCGGCAGCCTGCGCCTGAGCT
GCGCCGCCAGCGGCTTCTTCATCACCAACAACTACTGGGGCTGGGTGCGCCAGGCCCCCGGCAAG
GGCCTGGAGTGGGTGGGCTACATCAGCTACAGCGGCAGCACCAGCTACAACCCCAGCCTGAAGAG
CCGCTTCACCATCAGCCGCGACACCAGCAAGAACACCTTCTACCTGCAGATGAACAGCCTGCGCG
CCGAGGACACCGCCGTGTACTACTGCGCCCGCACCGGCAGCAGCGGCTACTTCGACTTCTGGGGC
CAGGGCACCCTGGTGACCGTGAGCAGCGCCTCCACCAAGGGCCCATCGGTCTTCCCCCTGGCACC
CTCCTCCAAGAGCACCTCTGGGGGCACAGCGGCCCTGGGCTGCCTGGTCAAGGACTACTTCCCCG
AACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACTGTGCCCTCTAGCAGCTTGGGCACC
CAGACCTACATCTGCAACGTGAATCACAAGCCCAGCAACACCAAGGTGGACAAGAAAGTTGAAC
CCAAATCTTGCGACAAAACTCACACATGCCCACCGTGCCCAGCACCTGAACTCCTGGGGGGACCG
TCAGTCTTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGACGGCGT
GGAGGTGCATAATGCCAAGACAAAGCCGCGGGAGGAGCAGTACGCCAGCACGTACCGTGTGGTC
AGCGTCCTCACCGTCCTGCACCAGGACTGGCTGAATGGCAAGGAGTACAAGTGCAAGGTCTCCAA
CAAAGCCCTCCCAGCCCCCATAGAGAAAACCATCTCCAAAGCCAAAGGGCAGCCCCGAGAACCA
CAGGTGTACACCCTGCCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTCTACAGCAAGCTCACCG
TGGACAAGAGCAGGTGGCAGCAGGGGAACGTCTTCTCATGCTCCGTGATGCATGAGGCTCTGCAC
AACCACTACACGCAGAAGAGCCTCTCCCTGTCTCCGGGTAAA
Etrolizumab LC (SEQ ID NO: 19) Nucleotide sequence:
GACATCCAGATGACCCAGAGCCCCAGCAGCCTGAGCGCCAGCGTGGGCGACCGCGTGACCATCAC
CTGCCGCGCCAGCGAGAGCGTGGACGACCTGCTGCACTGGTACCAGCAGAAGCCCGGCAAGGCC
CCCAAGCTGCTGATCAAGTACGCCAGCCAGAGCATCAGCGGCGTGCCCAGCCGCTTCAGCGGCAG
CGGCAGCGGCACCGACTTCACCCTGACCATCAGCAGCCTGCAGCCCGAGGACTTCGCCACCTACT
ACTGCCAGCAGGGCAACAGCCTGCCCAACACCTTCGGCCAGGGCACCAAGGTGGAGATCAAGCG
CACAGTGGCAGCCCCCAGCGTCTTCATTTTTCCCCCTTCCGATGAACAGCTGAAGTCCGGCACTGC
TTCTGTGGTCTGTCTGCTGAACAATTTCTATCCCAGAGAGGCCAAGGTGCAGTGGAAAGTGGACA
ACGCTCTGCAGTCCGGCAACAGCCAGGAGAGTGTGACCGAACAGGATAGTAAGGACAGCACATAT
TCTCTGTCTAGTACCCTGACACTGAGTAAGGCAGATTACGAGAAGCACAAAGTGTATGCCTGCGAA
GTCACTCATCAGGGACTGTCAAGCCCCGTGACCAAGAGCTTCAACCGGGGCGAGTGT
ATF-L1 LC (SEQ ID NO: 20) Nucleotide sequence:
GACATCCAGATGACCCAGAGCCCCAGCAGCCTGAGCGCCAGCGTGGGCGACCGCGTGACCATCAC
CTGCCGCGCCAGCGAGAGCGGCGGAAGCGGAGCAAAGCTCGCCGCACTGAAAGCCAAGCTGGCC
GCTCTGAAGGGAGGTGGCGGGAGCGCCGCTGCAATCTCCTGCGTCGGCAGCCCCGAATGTCCTCC
CAAGTGCCGGGCTCAGGGATGCAAGAACGGCAAGTGTATGAACCGGAAGTGCAAGTGCTACTATT
GCGGCGGAGGTGGGAGTGAACTGGCCGCACTGGAAGCTGAGCTGGCTGCCCTCGAAGCTGGAGG
CTCTGGACTGCTGCACTGGTACCAGCAGAAGCCCGGCAAGGCCCCCAAGCTGCTGATCAAGTACG
CCAGCCAGAGCATCAGCGGCGTGCCCAGCCGCTTCAGCGGCAGCGGCAGCGGCACCGACTTCACC
CTGACCATCAGCAGCCTGCAGCCCGAGGACTTCGCCACCTACTACTGCCAGCAGGGCAACAGCCT
GCCCAACACCTTCGGCCAGGGCACCAAGGTGGAGATCAAGCGCACAGTGGCAGCCCCCAGCGTC
TTCATTTTTCCCCCTTCCGATGAACAGCTGAAGTCCGGCACTGCTTCTGTGGTCTGTCTGCTGAACA
ATTTCTATCCCAGAGAGGCCAAGGTGCAGTGGAAAGTGGACAACGCTCTGCAGTCCGGCAACAGC
CAGGAGAGTGTGACCGAACAGGATAGTAAGGACAGCACATATTCTCTGTCTAGTACCCTGACACTG
AGTAAGGCAGATTACGAGAAGCACAAAGTGTATGCCTGCGAAGTCACTCATCAGGGACTGTCAAG
CCCCGTGACCAAGAGCTTCAACCGGGGCGAGTGT
ATF-L2 LC (SEQ ID NO: 21) Nucleotide sequence:
GACATCCAGATGACCCAGAGCCCCAGCAGCCTGAGCGCCAGCGTGGGCGACCGCGTGACCATCAC
CTGCCGCGCCAGCGAGAGCGTGGACGACCTGCTGCACTGGTACCAGCAGAAGCCCGGCAAGGCC
CCCAAGCTGCTGATCAAGTACGCCAGCCAGAGCATCAGCGGCGGAAGCGGAGCAAAGCTCGCCG
CACTGAAAGCCAAGCTGGCCGCTCTGAAGGGAGGTGGCGGGAGCGCCGCTGCAATCTCCTGCGTC
GGCAGCCCCGAATGTCCTCCCAAGTGCCGGGCTCAGGGATGCAAGAACGGCAAGTGTATGAACCG
GAAGTGCAAGTGCTACTATTGCGGCGGAGGTGGGAGTGAACTGGCCGCACTGGAAGCTGAGCTGG
CTGCCCTCGAAGCTGGAGGCTCTGGAAGCCGCTTCAGCGGCAGCGGCAGCGGCACCGACTTCACC
CTGACCATCAGCAGCCTGCAGCCCGAGGACTTCGCCACCTACTACTGCCAGCAGGGCAACAGCCT
GCCCAACACCTTCGGCCAGGGCACCAAGGTGGAGATCAAGCGCACAGTGGCAGCCCCCAGCGTC
TTCATTTTTCCCCCTTCCGATGAACAGCTGAAGTCCGGCACTGCTTCTGTGGTCTGTCTGCTGAACA
ATTTCTATCCCAGAGAGGCCAAGGTGCAGTGGAAAGTGGACAACGCTCTGCAGTCCGGCAACAGC
CAGGAGAGTGTGACCGAACAGGATAGTAAGGACAGCACATATTCTCTGTCTAGTACCCTGACACTG
AGTAAGGCAGATTACGAGAAGCACAAAGTGTATGCCTGCGAAGTCACTCATCAGGGACTGTCAAG
CCCCGTGACCAAGAGCTTCAACCGGGGCGAGTGT
ATF-L3 LC (SEQ ID NO: 22) Nucleotide sequence:
GACATCCAGATGACCCAGAGCCCCAGCAGCCTGAGCGCCAGCGTGGGCGACCGCGTGACCATCAC
CTGCCGCGCCAGCGAGAGCGTGGACGACCTGCTGCACTGGTACCAGCAGAAGCCCGGCAAGGCC
CCCAAGCTGCTGATCAAGTACGCCAGCCAGAGCATCAGCGGCGTGCCCAGCCGCTTCAGCGGCAG
CGGCAGCGGCACCGACTTCACCCTGACCATCAGCAGCCTGCAGCCCGAGGACTTCGCCACCTACT
ACTGCCAGCAGGGCGGCGGAAGCGGAGCAAAGCTCGCCGCACTGAAAGCCAAGCTGGCCGCTCT
GAAGGGAGGTGGCGGGAGCGCCGCTGCAATCTCCTGCGTCGGCAGCCCCGAATGTCCTCCCAAGT
GCCGGGCTCAGGGATGCAAGAACGGCAAGTGTATGAACCGGAAGTGCAAGTGCTACTATTGCGGC
GGAGGTGGGAGTGAACTGGCCGCACTGGAAGCTGAGCTGGCTGCCCTCGAAGCTGGAGGCTCTG
GAAACACCTTCGGCCAGGGCACCAAGGTGGAGATCAAGCGCACAGTGGCAGCCCCCAGCGTCTT
CATTTTTCCCCCTTCCGATGAACAGCTGAAGTCCGGCACTGCTTCTGTGGTCTGTCTGCTGAACAAT
TTCTATCCCAGAGAGGCCAAGGTGCAGTGGAAAGTGGACAACGCTCTGCAGTCCGGCAACAGCCA
GGAGAGTGTGACCGAACAGGATAGTAAGGACAGCACATATTCTCTGTCTAGTACCCTGACACTGAG
TAAGGCAGATTACGAGAAGCACAAAGTGTATGCCTGCGAAGTCACTCATCAGGGACTGTCAAGCC
CCGTGACCAAGAGCTTCAACCGGGGCGAGTGT
ATF-H1 HC (SEQ ID NO: 23) Nucleotide sequence:
GAGGTGCAGCTGGTGGAGAGCGGCGGCGGCCTGGTGCAGCCCGGCGGCAGCCTGCGCCTGAGCT
GCGCCGCCAGCGGCGGCGGAAGCGGAGCAAAGCTCGCCGCACTGAAAGCCAAGCTGGCCGCTCT
GAAGGGAGGTGGCGGGAGCGCCGCTGCAATCTCCTGCGTCGGCAGCCCCGAATGTCCTCCCAAGT
GCCGGGCTCAGGGATGCAAGAACGGCAAGTGTATGAACCGGAAGTGCAAGTGCTACTATTGCGGC
GGAGGTGGGAGTGAACTGGCCGCACTGGAAGCTGAGCTGGCTGCCCTCGAAGCTGGAGGCTCTG
GATACTGGGGCTGGGTGCGCCAGGCCCCCGGCAAGGGCCTGGAGTGGGTGGGCTACATCAGCTAC
AGCGGCAGCACCAGCTACAACCCCAGCCTGAAGAGCCGCTTCACCATCAGCCGCGACACCAGCA
AGAACACCTTCTACCTGCAGATGAACAGCCTGCGCGCCGAGGACACCGCCGTGTACTACTGCGCC
CGCACCGGCAGCAGCGGCTACTTCGACTTCTGGGGCCAGGGCACCCTGGTGACCGTGAGCAGCGC
CTCCACCAAGGGCCCATCGGTCTTCCCCCTGGCACCCTCCTCCAAGAGCACCTCTGGGGGCACAG
CGGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGC
AGCGTGGTGACTGTGCCCTCTAGCAGCTTGGGCACCCAGACCTACATCTGCAACGTGAATCACAA
GCCCAGCAACACCAAGGTGGACAAGAAAGTTGAACCCAAATCTTGCGACAAAACTCACACATGC
CCACCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGACAAAGCCG
CGGGAGGAGCAGTACGCCAGCACGTACCGTGTGGTCAGCGTCCTCACCGTCCTGCACCAGGACTG
GCTGAATGGCAAGGAGTACAAGTGCAAGGTCTCCAACAAAGCCCTCCCAGCCCCCATAGAGAAAA
CCATCTCCAAAGCCAAAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTATCCCAGCGACATCGC
CGTGGAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCCT
GTCTCCGGGTAAA
ATF-H2 HC (SEQ ID NO: 24) Nucleotide sequence:
GAGGTGCAGCTGGTGGAGAGCGGCGGCGGCCTGGTGCAGCCCGGCGGCAGCCTGCGCCTGAGCT
GCGCCGCCAGCGGCTTCTTCATCACCAACAACTACTGGGGCTGGGTGCGCCAGGCCCCCGGCAAG
GGCCTGGAGTGGGTGGGCTACATCAGCTACAGCGGCAGCACCAGCTACAACGGCGGAAGCGGAG
CAAAGCTCGCCGCACTGAAAGCCAAGCTGGCCGCTCTGAAGGGAGGTGGCGGGAGCGCCGCTGC
AATCTCCTGCGTCGGCAGCCCCGAATGTCCTCCCAAGTGCCGGGCTCAGGGATGCAAGAACGGCA
AGTGTATGAACCGGAAGTGCAAGTGCTACTATTGCGGCGGAGGTGGGAGTGAACTGGCCGCACTG
GAAGCTGAGCTGGCTGCCCTCGAAGCTGGAGGCTCTGGAAGCCGCTTCACCATCAGCCGCGACAC
CAGCAAGAACACCTTCTACCTGCAGATGAACAGCCTGCGCGCCGAGGACACCGCCGTGTACTACT
GCGCCCGCACCGGCAGCAGCGGCTACTTCGACTTCTGGGGCCAGGGCACCCTGGTGACCGTGAGC
AGCGCCTCCACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGAACT
CAGGCGCCCTGACCAGCGGCGTGCACACCTTCCCGGCTGTCCTACAGTCCTCAGGACTCTACTCCC
TCAGCAGCGTGGTGACTGTGCCCTCTAGCAGCTTGGGCACCCAGACCTACATCTGCAACGTGAATC
ACAAGCCCAGCAACACCAAGGTGGACAAGAAAGTTGAACCCAAATCTTGCGACAAAACTCACAC
ATGCCCACCGTGCCCAGCACCTGAACTCCTGGGGGGACCGTCAGTCTTCCTCTTCCCCCCAAAACC
CAAGGACACCCTCATGATCTCCCGGACCCCTGAGGTCACATGCGTGGTGGTGGACGTGAGCCACG
AAGACCCTGAGGTCAAGTTCAACTGGTACGTGGACGGCGTGGAGGTGCATAATGCCAAGACAAAG
CCGCGGGAGGAGCAGTACGCCAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATAGAGA
AAACCATCTCCAAAGCCAAAGGGCAGCCCCGAGAACCACAGGTGTACACCCTGCCCCCATCCCGG
GAGGAGATGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCTG
GACTCCGACGGCTCCTTCTTCCTCTACAGCAAGCTCACCGTGGACAAGAGCAGGTGGCAGCAGGG
GAACGTCTTCTCATGCTCCGTGATGCATGAGGCTCTGCACAACCACTACACGCAGAAGAGCCTCTC
CCTGTCTCCGGGTAAA
ATF-H3 HC (SEQ ID NO: 25) Nucleotide sequence:
GAGGTGCAGCTGGTGGAGAGCGGCGGCGGCCTGGTGCAGCCCGGCGGCAGCCTGCGCCTGAGCT
GCGCCGCCAGCGGCTTCTTCATCACCAACAACTACTGGGGCTGGGTGCGCCAGGCCCCCGGCAAG
GGCCTGGAGTGGGTGGGCTACATCAGCTACAGCGGCAGCACCAGCTACAACCCCAGCCTGAAGAG
CCGCTTCACCATCAGCCGCGACACCAGCAAGAACACCTTCTACCTGCAGATGAACAGCCTGCGCG
CCGAGGACACCGCCGTGTACTACTGCGCCCGCGGCGGAAGCGGAGCAAAGCTCGCCGCACTGAA
AGCCAAGCTGGCCGCTCTGAAGGGAGGTGGCGGGAGCGCCGCTGCAATCTCCTGCGTCGGCAGCC
CCGAATGTCCTCCCAAGTGCCGGGCTCAGGGATGCAAGAACGGCAAGTGTATGAACCGGAAGTGC
AAGTGCTACTATTGCGGCGGAGGTGGGAGTGAACTGGCCGCACTGGAAGCTGAGCTGGCTGCCCT
CGAAGCTGGAGGCTCTGGAGGCTACTTCGACTTCTGGGGCCAGGGCACCCTGGTGACCGTGAGCA
GCGCCTCCACCAAGGGCCCATCGGTCTTCCCCCTGGCACCCTCCTCCAAGAGCACCTCTGGGGGC
ACAGCGGCCCTGGGCTGCCTGGTCAAGGACTACTTCCCCGAACCGGTGACGGTGTCGTGGAACTC
AGGCGCCCTGACCAGCGGCGTGCACACCTTCCCGGCTGTCCTACAGTCCTCAGGACTCTACTCCCT
CAGCAGCGTGGTGACTGTGCCCTCTAGCAGCTTGGGCACCCAGACCTACATCTGCAACGTGAATCA
CAAGCCCAGCAACACCAAGGTGGACAAGAAAGTTGAACCCAAATCTTGCGACAAAACTCACACA
TGCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGC
CGCGGGAGGAGCAGTACGCCAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCCCCCATAGAGAA
AACCATCTCCAAAGCCAAAGGGCAGCCCCGAGAACCACAGGTGTACACCCTGCCCCCATCCCGGG
AGGAGATGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGG
ACTCCGACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCTCTCC
CTGTCTCCGGGTAAA
Vm24 (SEQ ID NO: 26) Nucleotide sequence:
GCCGCTGCAATCTCCTGCGTCGGCAGCCCCGAATGTCCTCCCAAGTGCCGGGCTCAGGGATGCAA
GAACGGCAAGTGTATGAACCGGAAGTGCAAGTGCTACTATTGC

### 2. Expression and Purification of Bispecific Binding Molecules

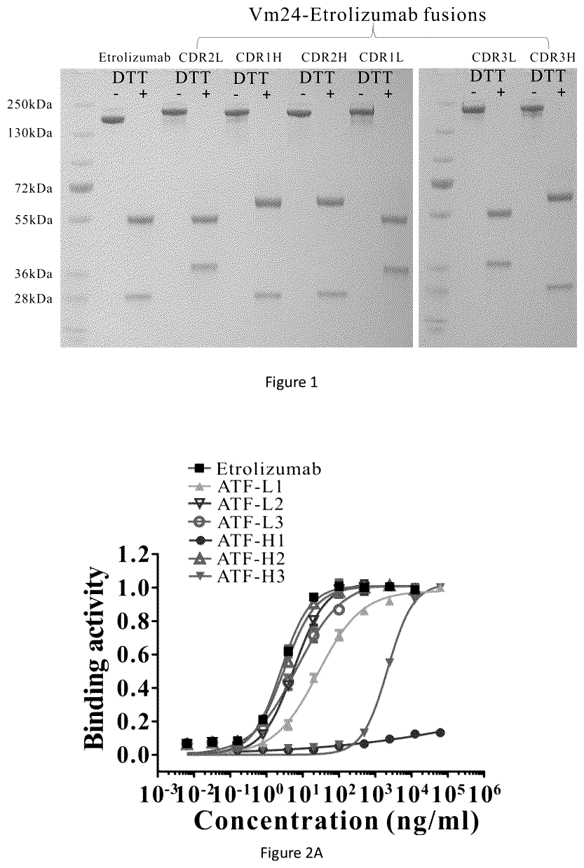

The constructed plasmids of bispecific binding molecules were transiently transfected into HEK293F cells for expression. The expression levels varied from 7 mg/L to 23 mg/L for different constructs. Under reducing agent DTT conditions, the molecular weights of HC and LC of bispecific binding molecules matched with expected (for example, when Vm24 is grafted into HC, then the molecular weight of corresponding HC is bigger), with the expression purity exceeding 90% (FIG. 1). The proteins were purified by size-exclusion chromatography (SEC), and then applied to binding and cell functional assays. Proteins for animal experiments underwent further endotoxin removal.

### 3. Affinity Testing of Bispecific Binding Molecules

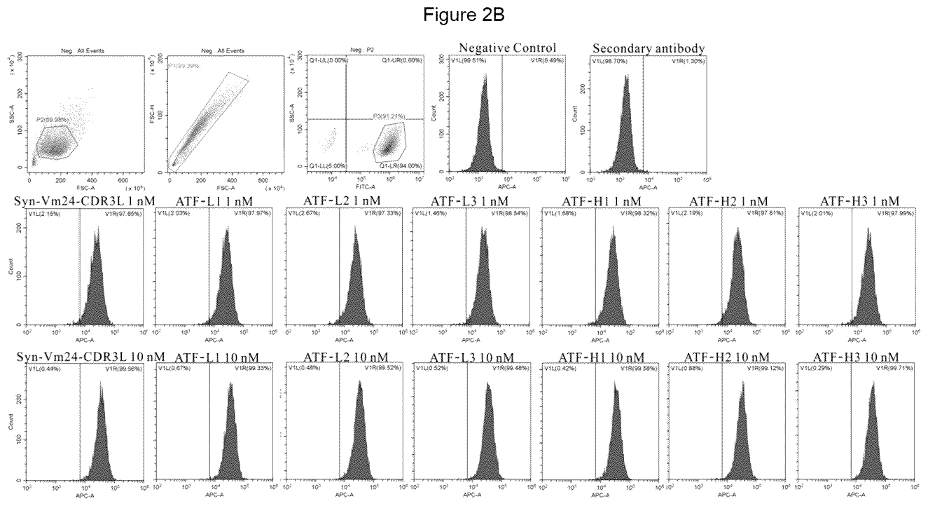

Recombinant protein α4β7 was coated on a 96-well plate at 100 ng per well, and enzyme-linked immunosorbent assay (ELISA) was applied to detect the binding capacity of the bispecific binding molecules. Results showed that the Kd values for α4β7 binding of ATF-H2(with HCDR2-grafted Vm24), ATF-L2(with LCDR2-grafted Vm24) and ATF-L3(with LCDR3-grafted Vm24) were 19.25±1.45 pM, 35.9±2.5 pM, and 40.82±6.6 pM, respectively, closely approaching the value of Kd=17.57±1.46 pM for etrolizumab. ATF-L1, with LCDR1-grafted Vm24, showed a decreased affinity (Kd=167.10±16.42 pM) than previous three bispecific molecules (ATF-H2, ATF-L2 and ATF-L3). The Kd of ATF-H3(with HCDR3-grafted Vm24) was 13.88±1.78 nM, while ATF-H1(with HCDR1-grafted Vm24) showed no binding for α4β7(FIG. 2A). Flow cytometry was applied to detect the binding of the bispecific binding molecules to Kv1.3 ion channel on stable cell line. The results indicated that the bispecific binding molecules showed comparable binding to that of Syn-Vm24-LCDR3 reported in previous literature (Wang. R. E. et al. PNAS 2016,113(41):11501-11506) at both concentrations of 1 nM and 10 nM (FIG. 2B).

### 4. In Vitro Activity of Bispecific Binding Molecules

### 4.1 Cell Adhesion Assay

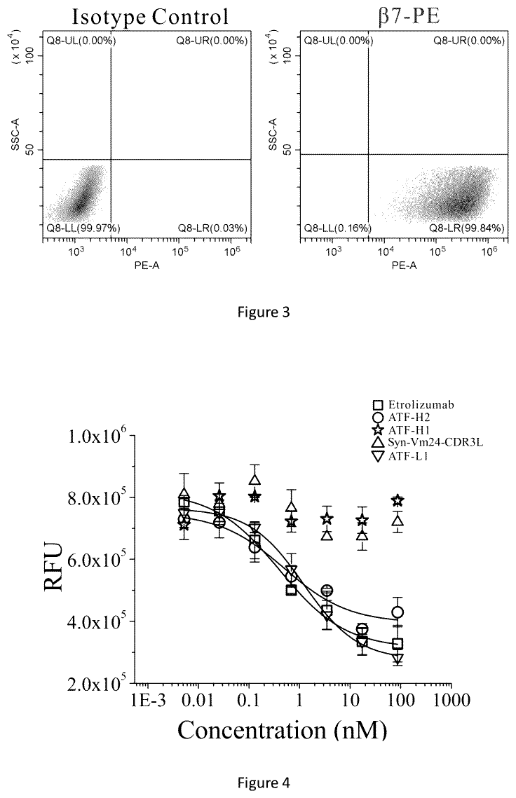

Human PBMCs were stimulated with PMA (5 ng/ml) and ionomycin (500 ng/ml) for 5-6 hours, and then transferred to normal medium. After 6-7 days, the cells were further stimulated as previously until β7-high T cells has been induced (in general, at least 3 stimulations needed) (FIG. 3). The cells were used for cell adhesion inhibition experiment.

Detailed experimental protocol was as follows: recombinant human MAdCAM-1 (200 ng/well) protein was coated in a 96-well plate at 4° C. overnight. The next day, the coating solution was aspirated and DPBS was applied 2-3 times for washing. Then DPBS containing 5% bovine serum albumin (BSA) was added to block for 1 hour at 37° C. The β7-high PBMCs (2*105 cells/well) were resuspended with detection solution (1 mM Mn2+, DMEM 0.1% BSA, 10 mM HEPES, pH 7), and incubated with different concentrations of the integrin β7 antibody, the bispecific binding molecules and control at 37° C. for 30 minutes (2*105 cells/well in 96-well plate), followed by another 1 h incubation to allow cells adhesion. Supernatant was aspirated, and DPBS were added for 2-3 times washing. Then 100 ul complete medium were added and equilibrated at room temperature for 30 minutes. CellTiter-Glo reagent was added to each well, shaken and mixed for 2 minutes to lyse the cells. After incubation of the mixture at room temperature for 10 minutes, relative fluorescence units (RFU) were recorded with a microplate reader, and the IC50 was then calculated.

Results showed that IC50 values were 0.06±0.02 μg/ml (0.42 nM±0.14 nM), 0.072±0.052 μg/ml (0.45 nM±0.33 nM) and 0.173±0.023 μg/ml (1.09 nM±0.14 nM) for etrolizumab, ATF-H2 and ATF-L1, respectively. ATF-H1 and Syn-Vm24-LCDR3 showed no significant inhibitory effect on the adhesion of β7-highT cells to MAdCAM-1 (FIG. 4).

### 4.2 Flipr Potassium Current Fluorescence Detection

The inhibitory effects of bispecific binding molecules on Kv1.3 current were assessed using the Flipr potassium current assay kit. The real-time fluorescence signal of Kv1.3 current was recorded by the Flipr high-throughput real-time fluorescence system. The IC50 values were calculated, and the inhibitory effects were compared. The results showed that the IC50 values for ATF-H1, ATF-H2, and ATF-L1 were 0.72±0.05 nM, 0.53±0.02 nM, and 0.70±0.06 nM, respectively, while etrolizumab showed no inhibitory effect on the Kv1.3 channel current (FIG. 5).

### 5. Selectivity of Bispecific Binding Molecules for Kv1.3 and β7

### 5.1 Construction of Kv1.3-High β7-High T Cell Model

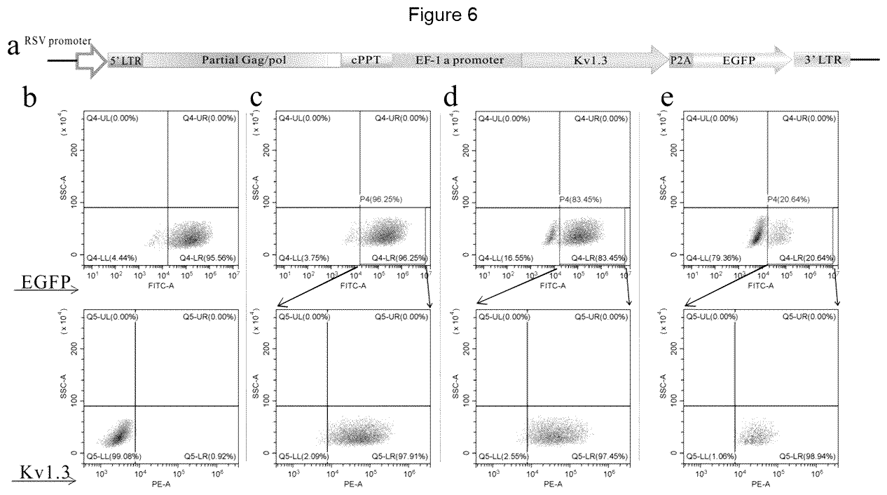

We hoped to select bispecific binding molecules that can bind Kv1.3-high TEM cells with binding preference, rather than all β7-high T cells. Among bispecific binding molecules constructed, ATF-L1, which was with moderate affinity for β7, high affinity for Kv1.3, as well as strong inhibitory activity on cell adhesion and Kv1.3 potassium current, was selected for binding selectivity test. Considering the relatively low expression of Kv1.3 on human T cells under physiological conditions, lentiviral vector with Kv1.3-EGFP high expression was constructed to better compare the selectivity of bispecific binding molecules to Kv1.3 and β7(FIG. 6A). Human PBMC cells were stimulated with PMA and ionomycin (as described in 4.1), and after more than three rounds of stimulation, the ratio of β7-high T cells were close to 100% (FIG. 3). The β7-high T cells were then infected with lentivirus with highly expressed Kv1.3-EGFP gene sequences, and the binding selectivity was tested when the infection rate was greater than 20% (FIG. 6B-6E).

### 5.2 Selectivity Experiment

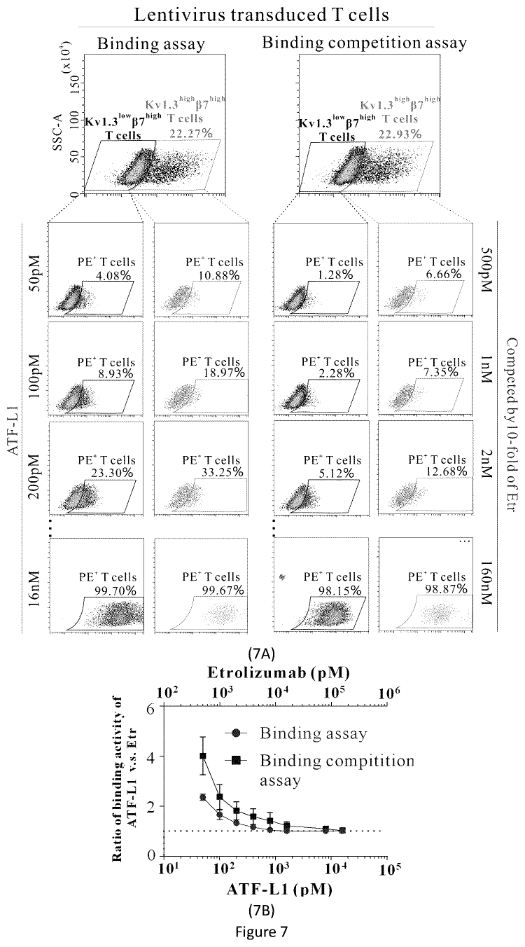

Binding selectivity was performed on Kv1.3-high β7-high T cells (>20%) and Kv1.3-low β7-high T cells by flow cytometry. PE/R-Phycoerythrin (Abcam) was covalently conjugated to ATF-L1, and then PE-labeled ATF-L1 at different concentrations was applied for human T cells binding. Cells were incubated with PE-labeled ATF-L1 at 4° C. for 1 hour, washed by 2-3 times to remove unbound antibody, and the binding capacity for Kv1.3-high β7-high T cells and Kv1.3-low β7-high T cells was assayed. Results showed that ATF-L1 at low and median concentration tended to bind Kv1.3-high β7-high T cells. The percentage of ATF-L1 bound to Kv1.3-high β7-high T cells was 2.4±0.1, 1.7±0.2, 1.3±0.1 and 1.2±0.1 times that bound on Kv1.3-low β7-high T cells at 50 pM, 100 pM, 200 pM and 400 pM, respectively (FIG. 7A left two columns, 7B). As the concentration decreased, the bispecific binding molecule was more inclined to bind to Kv1.3-high β7-high T cells. At 50 pM, the binding mobility with Kv1.3-high β7-high T cells was more than twice that with Kv1.3-low β7-high T cells. As the concentration increased, this binding preference gradually weakened. The reason may be that the proportion of T cells with high β7 expression was close to 100%, while T cells with high Kv1.3 expression was only 20%. Therefore, at high concentrations, a huge number of PE-labeled ATF bound to β7 on T cells, thus masking its preference binding to Kv1.3. To further test this notion, competition experiment was performed: T cells were incubated with different concentrations of PE-labeled ATF-L1 at 4° C. for 30 minutes, and washed with DPBS. Then 10-fold concentration of unlabeled etrolizumab was added and incubated at 4° C. for 1 hour. After washed 2-3 times with DPBS followed by resuspension, the fluorescence mobility of PE was detected by flow cytometry, and compared with that in the group without etrolizumab. Results showed that when comparing with Kv1.3-high β7-high T cells, PE-labeled ATF-L1 bound on Kv1.3-low β7-high T cells was more likely to be completed by etrolizumab as can be seen from the reduced fluorescence migration of PE (see two columns on the right in FIG. 7A). At 50 pM, the binding rate of PE-labeled ATF-L1 to Kv1.3-high β7-high T cells was 4 times that of Kv1.3-low β7-high T cells (FIG. 7B). These results show that the binding of ATF-1 for Kv1.3 were more selective than for β7, making it easier to bind to T cells with high Kv1.3 expression.

### 6. In Vivo Activity of Bispecific Binding Molecules in Animal Models

Human T cells infected with lentivirus, in which the rate of β7 expression was close to 100% and Kv1.3 expression was 10-20%, were adoptively transferred into DSS-induced inflammatory colitis model mice (Balb/c nude mice) or control mice via tail vein injection. The inhibitory effects of ATF-L1 on the migration of transferred human T cells to the colon tissues of the mice were then studied.

### 6.1 Establishment of DSS Inflammatory Colitis Model

7-8 week old Balb/c nude mice were used for modeling. Mice were divided into two groups based on body weight, namely the blank control group and the DSS model group. The mice in the blank control group were given normal drinking water every day, while the mice in the DSS model group were given drinking water containing 2% DSS every day for 5-7 days. Body weight changes and disease activity index (DAI) scores were recorded daily.

### 6.2 Adoptive Transfer of Human T Cells

1 mg of ATF-L1, etrolizumab or PBS were administered through the tail vein on the day before the adoptive transfer of cells. On the detection day, human T cells were labeled with Celltrace Yellow (Invitrogen), and then incubated in complete medium containing 200 pM ATF-L1, etrolizumab or PBS at 37° C. and 5% CO 2 for 1 hours. Before the adoptive transfer, 200 ul of peripheral blood was collected from the orbits of mice in each group as blank control. Hochst 33324 (Abeam) was injected into the tail vein to label the cell nuclei. After 30 minutes, 2×10⁶ human T cells labeled with Celltrace Yellow were adoptively transferred into mice through the tail vein. After 30 minutes, blood was collected through the orbit, and Texas Red dextran 70000 (Invitrogen) were injected through the tail vein for in vivo two-photon imaging of mouse colon tissue.

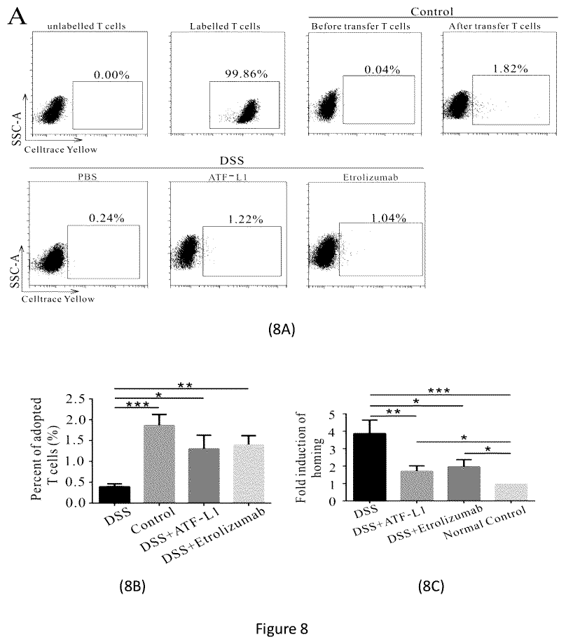

Peripheral blood collected before and after the adoptive transfer of cells was lysed with reagent from eBioscience™, and then subjected for flow cytometry analysis. The results showed that the proportion of human T cells in PBMCs from the DSS model group was 0.439±0.107%, which was significantly lower than that of other groups, indicating that the transferred cells may migrate to the site of intestinal inflammation; the proportion of human T cells in PBMCs from the blank control group was 1.987±0.326% (p<0.001); in group with DSS plus ATF-L1 treatment, the proportion of human T cells was 1.3±0.0.3% (p<0.05); while the proportion of human T cells in group with DSS plus Etrolizumab treatment was 1.4±0.2% (p<0.01) (FIG. 8A-C).

6.3 Imaging of Infiltrated Human T Cells with High Kv1.3 Expression in Colon Tissues

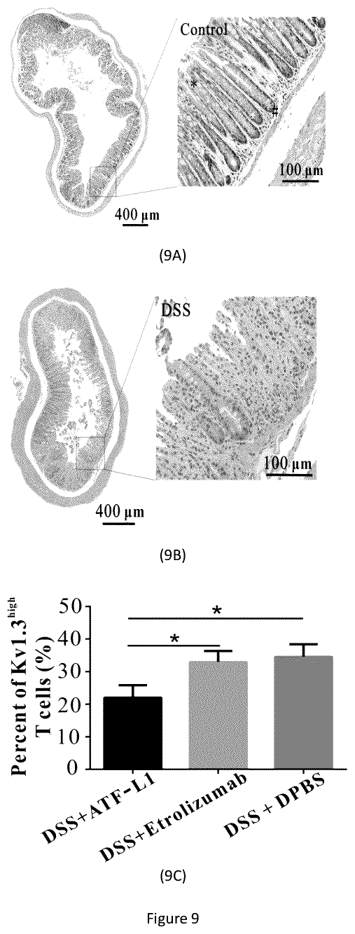

According to method reported in the literature (J Vis Exp.2012; (60):3678), the colon tissues of animals in each group were taken out, washed with saline, cut into 0.5-1 cm segments, and then fixed with 4% paraformaldehyde for 24 hours. Sections with a thickness of 100 μm were obtained with a vibrating microtome (Leica VT1000S), and then applied to imaging with a Fv3000 (Olympus) fluorescent confocal microscope for analysis of the number of Kv1.3 high-expressing infiltrated T cells in each group. At the same time, 4 μm sections in paraffin were made for HE staining. FIG. 9A-B showed that structure of the tissue sections from the DSS model animals displayed significant changes in comparison to that from the blank control group. The changes included destruction of the villi structure, a large number of immune cell infiltration, a large number of goblet cells, and lack of crypts. structure etc. The confocal imaging showed that a large number of infiltrating human T cells appeared in the lamina propria of the colonic mucosa in the DSS group in comparison to that DSS plus drugs treatment group. The number of Kv1.3 high-expressing T cells in infiltrated cells in DSS plus ATF-L1 treatment group was significantly lower than that in DSS plus etrolizumab treatment group and DSS model groups (FIG. 9C, p<0.05). The results showed that the bispecific binding molecule can specifically inhibit the infiltration of human Kv1.3 high-expressing T cell into inflammatory tissues in vivo.

### 7. Safety and Efficacy of Bispecific Binding Molecules

7-9 week-old Balb/c mice were randomly divided into seven groups for safety and efficacy studies. Various doses of ATF-L1 or etrolizumab were administered to assess their effects on weight changes, disease activity index, and colon tissue length in DSS-induced colitis model mice. The results indicated that ATF-L1 effectively alleviated weight loss and improved disease progression in the DSS-induced colitis model mice.

7-9 week-old Balb/c mice were randomly divided into 7 groups basing on body weight: ATL-1 10 mg/kg (low dose) group, ATL-1 15 mg/kg (high dose) group, etrolizumab 9.1 mg/kg (low dose) group, etrolizumab 13.6 mg/kg (high dose) group, panitumumab 14 mg/kg group, DSS model group, and the blank control group (DPBS group). All mice in DSS model group or DSS plus drugs treatment groups were given drinking water containing 2% DSS for 5 days and then normal drinking water without DSS for another 4 days, while the DPBS group were only given normal drinking water without DSS. Body weight changes and disease activity index (DAI) scores were recorded every day, and the grading criteria were shown in Table 1. All mice were sacrificed on the 9th day, and the colon tissues were taken for further analysis.

TABLE 1
Detailed grading criteria for DAI
Clinical activity score

| Score | Weight | Stool consistency | Blood in stool |
|---|---|---|---|
| 0 | 0 | normal | No blood |
| 1 | 1-5% | soft | |
| 2 | 5-10% | very soft | Visual pellet bledding |
| 3 | 10-20% | diarrhea | |
| 4 | >20% | | gross bleeding, blood around anus |

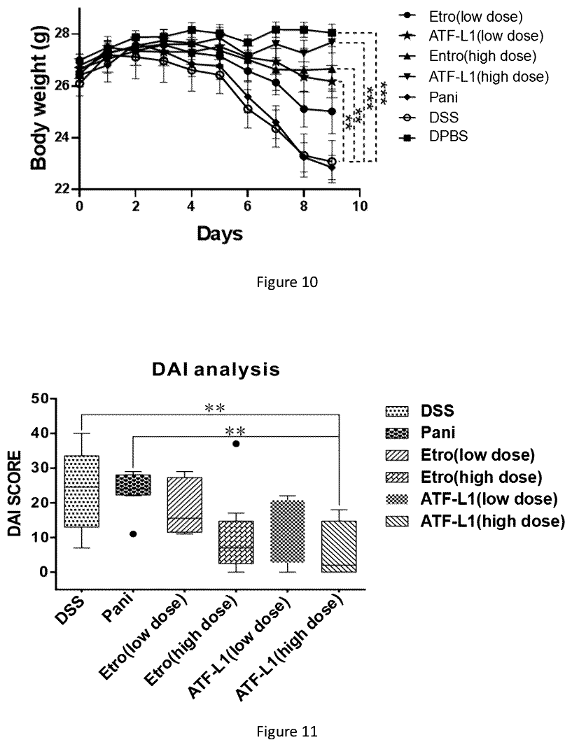

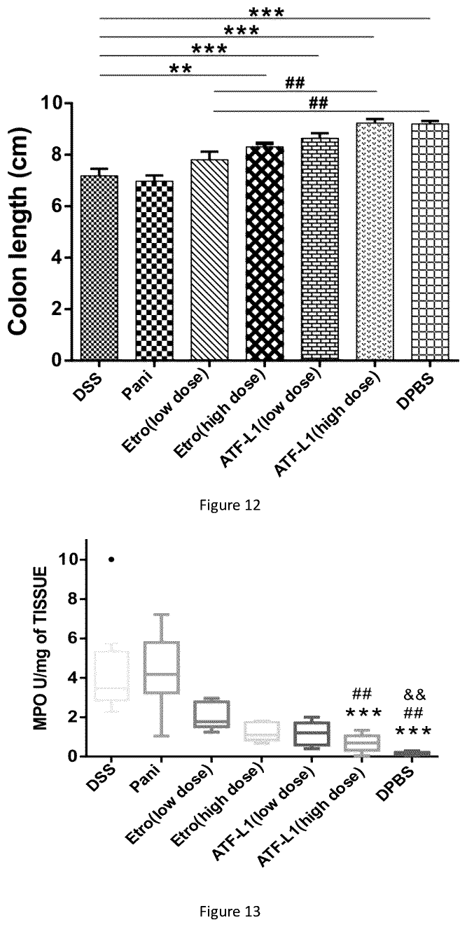

Results showed that the body weight changes in all three groups (ATF-L1 10 mg/kg, ATF-L1 15 mg/kg and etrolizumab 13.6 mg/kg) were significantly different from that in DSS model group (FIG. 10), indicating that DSS could effectively alleviate the weight loss of mice in all three groups (ATF-L1 10 mg/kg, ATF-L1 15 mg/kg and etrolizumab 13.6 mg/kg).

The DAI scores of each mouse in each group were summed up, and the differences among the groups were compared. As shown in FIG. 11, significant differences were observed not only between ATF-L1 15 mg/kg group and DSS model group, but also between ATF-L1 15 mg/kg group and panitumumab group (14 mg/kg) (**, P<0.01, one-way Anova test), suggesting that high dose of ATF-L1significantly improved the DSS-induced colonic inflammatory disease process in mice.

On the 9th day, the animals were sacrificed by injecting excess chloral hydrate, the colon tissues were dissected, and the length of colons was measured. As shown in FIG. 13, the colon length in all three groups (ATF-L1 10 mg/kg, ATF-L1 15 mg/kg and etrolizumab 13.6 mg/kg) were significantly different from that in DSS model group or in DSS plus panitumumab treatment group (*, P<0.05; ***, P<0.001; one-way Anova test). Significant difference between the high-dose ATF-L1 group (15 mg/kg) and the DPBS group was also observed (***, P<0.001, one-way Anova test). No significant difference was observed not only between ATF-L1 groups (10 mg/kg and 15 mg/kg) and DPBS group, but also between DSS plus Etrolizumab treatment group (13.6 mg/kg) and DPBS group. Results above implied that the 10 mg/kg and 15 mg/kg of ATF-L1 as well as 13.6 mg/kg of Etrolizumab can significantly improve the shortening of colon tissue in animals caused by DSS.

What is claimed is:

1. A bispecific binding molecule comprising:
A) an inhibitory peptide targeting for Kv1.3 potassium ion channel;
B) an antibody specifically binding to integrin β7;
wherein the inhibitory peptide is fused to the antibody directly or through a linker peptide;
wherein the amino acid sequence of A) peptide is grafted into the amino acid sequences of the heavy chain variable region (VH) and/or light chain variable region (VL) of B) antibody.

2. (canceled)

3. (canceled)

4. The bispecific binding molecule according to claim 1, wherein the amino acid sequence of A) peptide is grafted into the amino acid sequences of the heavy chain complementarity-determining region (HCDR) and/or light chain complementarity-determining region (LCDR) of B) antibody;
preferably, wherein the amino acid sequence of A) peptide is grafted into the amino acid sequences of HCDR1 or HCDR3 of B) antibody;
or the amino acid sequence of A) peptide is grafted into the amino acid sequences of LCDR1 or LCDR3 of B) antibody.

5. (canceled)

6. (canceled)

7. The bispecific binding molecule according to claim 1, wherein the amino acid sequence of A) peptide is grafted into the amino acid sequences of the framework region (FR) of B) antibody;
preferably, wherein the amino acid sequence of A) peptide is grafted into the amino acid sequences of heavy chain FR3 and/or light chain FR3 of B) antibody.

8. (canceled)

9. A bispecific binding molecule according to claim 1, wherein the molecule comprises heavy chain (HC) peptide and light chain (LC) peptide, wherein:
the HC peptide contains the constructs of A) peptide grafted into the heavy chain of B) antibody, and the LC peptide contains the light chain of B) antibody;
the LC peptide contains the constructs of A) peptide grafted into the light chain of B) antibody, and the HC peptide contains the heavy chain of B) antibody; or
the HC peptide contains the constructs of A) peptide grafted into the heavy chain of B) antibody, and the LC peptide contains the constructs of A) peptide grafted into the light chain of B) antibody.

10. A bispecific binding molecule according to claim 1, wherein A) peptide is a toxin peptide derived from the Mexican scorpion (Vaejovis mexicanus).

11. A bispecific binding molecule according to claim 1, wherein A) peptide is Vm24.

12. A bispecific binding molecule according to claim 1, wherein A) peptide has an amino acid sequence as shown in SEQ ID NO: 9 (AAAISCVGSPECPPKCRAQGCKNGKCMNRKCKCYYC).

13. A bispecific binding molecule according to claim 1, wherein B) antibody has the amino acid sequences of LCDR1 as shown in SEQ ID NO: 10, LCDR2 as shown in SEQ ID NO: 11, LCDR3 as shown in SEQ ID NO: 12, HCDR1 as shown in SEQ ID NO: 13, HCDR2 as shown in SEQ ID NO: 14, and HCDR3 as shown in SEQ ID NO: 15.

14. A bispecific binding molecule according to claim 1, wherein B) antibody has the VH sequence from 1-117 amino acid residues as shown in SEQ ID NO: 1 and the VL sequence from 1-107 amino acid residues as shown in SEQ ID NO: 2.

15. A bispecific binding molecule according to claim 1, wherein the linker peptide has a sequence as shown in
(SEQ ID NO: 16)
GGSGAKLAALKAKLAALKGGGGS
or
(SEQ ID NO: 17)
GGGGSELAALEAELAALEAGGSG.

16. The bispecific binding molecule according to claim 15, comprising:
HC sequences selected from SEQ ID NOs: 1, 6, 7, 8, and
LC sequences selected from SEQ ID NOs: 2, 3, 4, 5.

17. The bispecific binding molecule according to claim 16, comprising:
HC sequences as shown in SEQ ID NO: 1 and LC sequences as shown in SEQ ID NO: 3;
HC sequences as shown in SEQ ID NO: 1 and LC sequences as shown in SEQ ID NO: 5;
HC sequences as shown in SEQ ID NO: 6 and LC sequences as shown in SEQ ID NO: 2;
HC sequences as shown in SEQ ID NO: 8 and LC sequences as shown in SEQ ID NO: 2;
HC sequences as shown in SEQ ID NO: 1 and LC sequences as shown in SEQ ID NO: 4; or
HC sequences as shown in SEQ ID NO: 7 and LC sequences as shown in SEQ ID NO: 2.

18. A method for modifying an antibody specific to integrin β7 to enhance its therapeutic efficacy, comprising grafting a targeted inhibitory peptide for Kv1.3 potassium ion channel into the VH and/or VL regions of the antibody;
wherein the antibody is the B) antibody according to claim 1, and the targeted inhibitory peptide for the Kv1.3 potassium ion channel is the A) peptide according to claim 1.

19. A method for providing a therapeutic agent of integrin β7 antibody, wherein the integrin β7 antibody comprising:
grafting a targeted inhibitory peptide for the Kv1.3 potassium ion channel into the VH and/or VL regions of the integrin β7 antibody to generate antibody variants;
screening for variant antibodies with higher selectivity for Kv1.3highβ7high T cells over Kv1.3lowβ7high T cells;
wherein the antibody is the B) antibody according to claim 1, and the targeted inhibitory peptide for the Kv1.3 potassium ion channel is the A) peptide according to claim 1.

20. (canceled)

21. A nucleic acid encoding the bispecific binding molecule according to claim 1.

22. An expression vector comprising the nucleic acid according to claim 21.

23. A host cell comprising the expression vector according to claim 22.

24. A method for producing the bispecific binding molecule according to claim 1, comprising: culturing the host cell which comprising an expression vector with a nucleic acid encoding the bispecific binding molecule under conditions suitable for the expression of the nucleic acid which encoding the bispecific binding molecule, and isolating the bispecific binding molecule.

25. A pharmaceutical composition comprising the bispecific binding molecule according to claim 1 and a pharmaceutically acceptable carrier.

26. A method for treating or preventing conditions related to Kv1.3 potassium ion channel and/or integrin β7, wherein the method comprises: administering a therapeutically effective amount of the bispecific binding molecule according to claim 1 to a subject diagnosed with the conditions; or administering a prophylactically effective amount of the bispecific binding molecule according to claim 1 to a subject for preventing the conditions;
preferably, wherein the conditions related to Kv1.3 potassium ion channel and/or integrin β7 comprises: inflammatory conditions, immune and proliferative diseases, rheumatoid arthritis (RA), ankylosing spondylitis, psoriatic arthritis, osteoarthritis, osteoporosis, uveitis, inflammatory fibrosis, scleroderma, pulmonary fibrosis, cirrhosis, inflammatory bowel disease, Crohn's disease, ulcerative colitis, asthma, allergic asthma, allergies, chronic obstructive pulmonary disease (COPD), multiple sclerosis, psoriasis, contact-mediated dermatitis, systemic lupus erythematosus (SLE) and other forms of lupus, diabetes, type I diabetes, obesity, cancer, lupus, restenosis, systemic sclerosis, scleroderma, glomerulonephritis, dry syndrome, inflammatory bone resorption, transplant rejection, or graft-versus-host disease

27. (canceled)


SEQUENCE LISTING (26 sequences)

SEQ ID NO: 1 - protein, 447 aa, synthetic construct
EVQLVESGGGLVQPGGSLRLSCAASGFFITNNYWGWVRQAPGKGLEWVGYISYSGSTSYNPSLKSRFTISRDTSKNTFYLQMNSLRAEDTAVYYCARTGSSGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASVGDRVTITCRASESVDDLLHWYQQKPGKAPKLLIKYASQSISGVPSRFSGSGSGTDFTLTISSLQPEDFATYYCQQGNSLPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 293 aa, synthetic construct
DIQMTQSPSSLSASVGDRVTITCRASESGGSGAKLAALKAKLAALKGGGGSAAAISCVGSPECPPKCRAQGCKNGKCMNRKCKCYYCGGGGSELAALEAELAALEAGGSGLLHWYQQKPGKAPKLLIKYASQSISGVPSRFSGSGSGTDFTLTISSLQPEDFATYYCQQGNSLPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 293 aa, synthetic construct
DIQMTQSPSSLSASVGDRVTITCRASESVDDLLHWYQQKPGKAPKLLIKYASQSISGGSGAKLAALKAKLAALKGGGGSAAAISCVGSPECPPKCRAQGCKNGKCMNRKCKCYYCGGGGSELAALEAELAALEAGGSGSRFSGSGSGTDFTLTISSLQPEDFATYYCQQGNSLPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 292 aa, synthetic construct
DIQMTQSPSSLSASVGDRVTITCRASESVDDLLHWYQQKPGKAPKLLIKYASQSISGVPSRFSGSGSGTDFTLTISSLQPEDFATYYCQQGGGSGAKLAALKAKLAALKGGGGSAAAISCVGSPECPPKCRAQGCKNGKCMNRKCKCYYCGGGGSELAALEAELAALEAGGSGNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 523 aa, synthetic construct
EVQLVESGGGLVQPGGSLRLSCAASGGGSGAKLAALKAKLAALKGGGGSAAAISCVGSPECPPKCRAQGCKNGKCMNRKCKCYYCGGGGSELAALEAELAALEAGGSGYWGWVRQAPGKGLEWVGYISYSGSTSYNPSLKSRFTISRDTSKNTFYLQMNSLRAEDTAVYYCARTGSSGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 525 aa, synthetic construct
EVQLVESGGGLVQPGGSLRLSCAASGFFITNNYWGWVRQAPGKGLEWVGYISYSGSTSYNGGSGAKLAALKAKLAALKGGGGSAAAISCVGSPECPPKCRAQGCKNGKCMNRKCKCYYCGGGGSELAALEAELAALEAGGSGSRFTISRDTSKNTFYLQMNSLRAEDTAVYYCARTGSSGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 525 aa, synthetic construct
EVQLVESGGGLVQPGGSLRLSCAASGFFITNNYWGWVRQAPGKGLEWVGYISYSGSTSYNPSLKSRFTISRDTSKNTFYLQMNSLRAEDTAVYYCARGGSGAKLAALKAKLAALKGGGGSAAAISCVGSPECPPKCRAQGCKNGKCMNRKCKCYYCGGGGSELAALEAELAALEAGGSGGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 36 aa, synthetic construct
AAAISCVGSPECPPKCRAQGCKNGKCMNRKCKCYYC

SEQ ID NO: 10 - protein, 6 aa, synthetic construct
ESVDDL

SEQ ID NO: 11 - protein, 5 aa, synthetic construct
KYASQ

SEQ ID NO: 12 - protein, 9 aa, synthetic construct
QQGNSLPNT

SEQ ID NO: 13 - protein, 6 aa, synthetic construct
FFITNN

SEQ ID NO: 14 - protein, 7 aa, synthetic construct
ISYSGST

SEQ ID NO: 15 - protein, 10 aa, synthetic construct
ARTGSSGYFD

SEQ ID NO: 16 - protein, 23 aa, synthetic construct
GGSGAKLAALKAKLAALKGGGGS

SEQ ID NO: 17 - protein, 23 aa, synthetic construct
GGGGSELAALEAELAALEAGGSG

SEQ ID NO: 18 - DNA, 1341 bp, synthetic construct
  source 1..1341: mol_type = other DNA
gaggtgcagctggtggagagcggcggcggcctggtgcagcccggcggcagcctgcgcctgagctgcgccgccagcggcttcttcatcaccaacaactactggggctgggtgcgccaggcccccggcaagggcctggagtgggtgggctacatcagctacagcggcagcaccagctacaaccccagcctgaagagccgcttcaccatcagccgcgacaccagcaagaacaccttctacctgcagatgaacagcctgcgcgccgaggacaccgccgtgtactactgcgcccgcaccggcagcagcggctacttcgacttctggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - DNA, 642 bp, synthetic construct
  source 1..642: mol_type = other DNA
gacatccagatgacccagagccccagcagcctgagcgccagcgtgggcgaccgcgtgaccatcacctgccgcgccagcgagagcgtggacgacctgctgcactggtaccagcagaagcccggcaaggcccccaagctgctgatcaagtacgccagccagagcatcagcggcgtgcccagccgcttcagcggcagcggcagcggcaccgacttcaccctgaccatcagcagcctgcagcccgaggacttcgccacctactactgccagcagggcaacagcctgcccaacaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 20 - DNA, 879 bp, synthetic construct
  source 1..879: mol_type = other DNA
gacatccagatgacccagagccccagcagcctgagcgccagcgtgggcgaccgcgtgaccatcacctgccgcgccagcgagagcggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggactgctgcactggtaccagcagaagcccggcaaggcccccaagctgctgatcaagtacgccagccagagcatcagcggcgtgcccagccgcttcagcggcagcggcagcggcaccgacttcaccctgaccatcagcagcctgcagcccgaggacttcgccacctactactgccagcagggcaacagcctgcccaacaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 21 - DNA, 879 bp, synthetic construct
  source 1..879: mol_type = other DNA
gacatccagatgacccagagccccagcagcctgagcgccagcgtgggcgaccgcgtgaccatcacctgccgcgccagcgagagcgtggacgacctgctgcactggtaccagcagaagcccggcaaggcccccaagctgctgatcaagtacgccagccagagcatcagcggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggaagccgcttcagcggcagcggcagcggcaccgacttcaccctgaccatcagcagcctgcagcccgaggacttcgccacctactactgccagcagggcaacagcctgcccaacaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 22 - DNA, 876 bp, synthetic construct
  source 1..876: mol_type = other DNA
gacatccagatgacccagagccccagcagcctgagcgccagcgtgggcgaccgcgtgaccatcacctgccgcgccagcgagagcgtggacgacctgctgcactggtaccagcagaagcccggcaaggcccccaagctgctgatcaagtacgccagccagagcatcagcggcgtgcccagccgcttcagcggcagcggcagcggcaccgacttcaccctgaccatcagcagcctgcagcccgaggacttcgccacctactactgccagcagggcggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggaaacaccttcggccagggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 23 - DNA, 1569 bp, synthetic construct
  source 1..1569: mol_type = other DNA
gaggtgcagctggtggagagcggcggcggcctggtgcagcccggcggcagcctgcgcctgagctgcgccgccagcggcggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggatactggggctgggtgcgccaggcccccggcaagggcctggagtgggtgggctacatcagctacagcggcagcaccagctacaaccccagcctgaagagccgcttcaccatcagccgcgacaccagcaagaacaccttctacctgcagatgaacagcctgcgcgccgaggacaccgccgtgtactactgcgcccgcaccggcagcagcggctacttcgacttctggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - DNA, 1575 bp, synthetic construct
  source 1..1575: mol_type = other DNA
gaggtgcagctggtggagagcggcggcggcctggtgcagcccggcggcagcctgcgcctgagctgcgccgccagcggcttcttcatcaccaacaactactggggctgggtgcgccaggcccccggcaagggcctggagtgggtgggctacatcagctacagcggcagcaccagctacaacggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggaagccgcttcaccatcagccgcgacaccagcaagaacaccttctacctgcagatgaacagcctgcgcgccgaggacaccgccgtgtactactgcgcccgcaccggcagcagcggctacttcgacttctggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 25 - DNA, 1575 bp, synthetic construct
  source 1..1575: mol_type = other DNA
gaggtgcagctggtggagagcggcggcggcctggtgcagcccggcggcagcctgcgcctgagctgcgccgccagcggcttcttcatcaccaacaactactggggctgggtgcgccaggcccccggcaagggcctggagtgggtgggctacatcagctacagcggcagcaccagctacaaccccagcctgaagagccgcttcaccatcagccgcgacaccagcaagaacaccttctacctgcagatgaacagcctgcgcgccgaggacaccgccgtgtactactgcgcccgcggcggaagcggagcaaagctcgccgcactgaaagccaagctggccgctctgaagggaggtggcgggagcgccgctgcaatctcctgcgtcggcagccccgaatgtcctcccaagtgccgggctcagggatgcaagaacggcaagtgtatgaaccggaagtgcaagtgctactattgcggcggaggtgggagtgaactggccgcactggaagctgagctggctgccctcgaagctggaggctctggaggctacttcgacttctggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - DNA, 108 bp, synthetic construct
  source 1..108: mol_type = other DNA
gccgctgcaatctcctgcgtcggcagccccgaatgtcctcccaagtgccgggctcagggatgcaagaacggcaagtgtatgaaccggaagtgcaagtgctactattgc